(19) United States
(12) Patent Application Publication
(10) Pub. No.: US 2023/0381423 A1
(43) Pub. Date: Nov. 30, 2023

(54) DRUG DELIVERY DEVICE ASSEMBLY AND ACCESSORY FOR DRUG DELIVERY DEVICE

(71) Applicant: AMGEN INC., Thousand Oaks, CA (US)

(72) Inventors: Jakob Halkjaer Pedersen, Frederiksberg (DK); Joshua Jay Dudman, Copenhagen (DK)

(21) Appl. No.: 18/032,765

(22) PCT Filed: Nov. 1, 2021
(86) PCT No.: PCT/US21/57507

§ 371 Date: Apr. 19, 2023

Related U.S. Application Data
(60) Provisional application No. 63/142,056, filed on Jan. 27, 2021.
(60) Provisional application No. 63/109,632, filed on Nov. 4, 2020.

(51) Int. Cl. A61M 5/32

(52) U.S. Cl. CPC A61M 5/3221; A61M 2205/581; A61M 2205/583; A61M 2205/582

(57) ABSTRACT

A drug delivery device assembly is provided, including an injector housing, a needle assembly, a drive assembly, and a needle shield indicator accessory. The injector housing may include a body with a proximal end, a distal end, and a longitudinal axis extending between the proximal end and the distal end, and a needle shield positioned adjacent to the distal end and movable between an extended position and a retracted position. The needle assembly is at least partially disposed within the body and may include a syringe barrel containing a medicament and a needle or a cannula. The drive assembly is at least partially disposed within the body and operably coupled with the needle assembly to urge the medicament through the needle or cannula during an injection sequence. The needle shield indicator accessory includes an accessory body configured to be selectively coupled with the injector housing and a needle shield indicator configured to indicate to a user whether the needle shield is in the retracted position.

### CROSS-REFERENCE TO RELATED APPLICATIONS

Priority is claimed to U.S. Provisional Patent Application No. 63/109,632, filed Nov. 4, 2020, and U.S. Provisional Patent Application No. 63/142,056, filed Jan. 27, 2021, the entire contents of each of which are hereby incorporated herein by reference.

### FIELD OF THE INVENTION

This disclosure generally relates to a drug delivery device assembly and accessory for a drug delivery device. More particularly, the disclosure generally relates to an assembly with a needle shield indicator accessory and/or a needle shield indicator accessory that includes an accessory body configured to be selectively coupled with the injector housing and a needle shield indicator configured to indicate to a user whether the needle shield is in the retracted position.

### BACKGROUND

Drugs are administered to treat a variety of conditions and diseases. Autoinjectors (e.g., pen style autoinjectors) and on-body injectors offer several benefits in delivery of medicaments such as drugs and/or therapeutics. One of the benefits can include simplicity of use, as compared with traditional methods of delivery using, for example, conventional syringes. Autoinjectors may be used to deliver many different medicaments with varying viscosities and/or desired volumes.

Many injectors include a needle shield that at least partially surrounds the injector needle and is and movable between an extended position and a retracted position. The needle shield often serves a dual function: protecting the user and others from inadvertent needle sticks and providing at least one of the required steps for initiating actuation. As an example, many injectors are initiated when the needle shield is moved into the retracted position, typically by pressing the needle shield against the users skin at a desired injection site. Some autoinjectors require an additional step, such as pressing an actuation button. In any case, some users may find it difficult to fully retract the needle shield by pressing it against their skin. As a more specific example, the needle shield may depress the users skin rather than the users skin depressing the needle shield, thereby delaying injection initiation or otherwise preventing or complicating a successful injection.

Users may also be instructed to and/or desire to observe the needle shield to determine when it has been fully retracted. As an example, the user may wish to know when the needle shield is retracted so they know when the injection is about to commence and/or so they know when to press the actuation button.

It may also be desirable for autoinjector users to maintain a particular force level and/or orientation during the injection. Autoinjector IFUs may instruct, encourage, or recommend such actions. For example, a user may desire to or be instructed to maintain a constant or baseline force and/or to maintain the autoinjector in an orientation perpendicular to the injection site during the injection sequence. These steps may increase the likelihood of a complete and successful injection and/or reduce pain or discomfort.

However, some autoinjector users may find it awkward, uncomfortable, or otherwise inconvenient to apply the desired force at the desired orientation, for example while also observing or otherwise ensuring that the needle shield has been properly retracted.

As described in more detail below, the present disclosure sets forth a drug delivery device assembly and an accessory for drug delivery devices, such as autoinjectors, that embodies advantageous alternatives to existing systems and methods, and that may address one or more of the challenges or needs mentioned herein, as well as provide other benefits and advantages.

### SUMMARY

A drug delivery device assembly is provided, including an injector housing, a needle assembly, a drive assembly, and a needle shield indicator accessory. The injector housing may include a body with a proximal end, a distal end, and a longitudinal axis extending between the proximal end and the distal end, and a needle shield positioned adjacent to the distal end and movable between an extended position and a retracted position. The needle assembly is at least partially disposed within the body and may include a syringe barrel containing a medicament and a needle or a cannula. The drive assembly is at least partially disposed within the body and operably coupled with the needle assembly to urge the medicament through the needle or cannula during an injection sequence. The needle shield indicator accessory includes an accessory body configured to be selectively coupled with the injector housing and a needle shield indicator configured to indicate to a user whether the needle shield is in the retracted position.

The needle shield indicator may include a feedback component configured to provide the user feedback when the needle shield is in the retracted position. The feedback component may be movable between a first position and a second position. The feedback component may provide the user with a visual indicator, an audible indicator, and/or a haptic indicator when the needle shield is in the retracted position.

The needle shield indicator may include a contact surface configured to contact the user, wherein the contact surface is movable between an extended position and a retracted position, and wherein the retracted position of the contact surface corresponds with the retracted position of the needle shield. The contact surface may be configured to trigger the feedback component when the contact surface is in the retracted position.

The accessory body may include a distal portion having a diameter significantly larger than a diameter of the needle shield.

The needle shield indicator may include an accessory reference feature, wherein the injector housing includes an injector reference feature, and wherein the accessory reference feature and the injector reference feature are aligned when the needle shield is in the retracted position. The accessory reference feature and the injector reference feature are not aligned when the needle shield is in the extended position.

The injector housing may include a drug viewing window and the injector reference feature may be defined by at least a portion of the drug viewing window. The accessory reference feature may be a cutout portion having a similar shape as the portion of the drug viewing window.

The accessory body may include a distal portion having a diameter significantly larger than a diameter of the needle shield.

A needle shield indicator accessory for a drug delivery device is also provided, including an accessory body configured to be selectively coupled with an injector housing and a needle shield indicator configured to indicate to a user whether an injector needle shield is in a retracted position.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description.

FIG. 1 is a front view of an exemplary drug delivery device that may be utilized with aspects of the present disclosure;

FIG. 2A is a front view of an exemplary drug delivery device accessory according to aspects of the present disclosure, including a needle shield indicator configured to indicate to a user whether the needle shield is in the retracted position;

FIG. 2B is a cross-sectional view of the needle shield indicator aid shown in FIG. 2A, coupled with an injector, to form an assembly according to aspects of the present disclosure;

FIG. 2C is an exploded view of the assembly shown in FIG. 2B;

FIG. 2D is view of the assembly shown in FIG. 2B in a first position;

FIG. 2E is view of the assembly shown in FIG. 2B in a second position;

FIG. 3A is a perspective view of another exemplary drug delivery device accessory according to aspects of the present disclosure, including a needle shield indicator configured to indicate to a user whether the needle shield is in the retracted position;

FIG. 3B is a view of the needle shield indicator aid shown in FIG. 3A, coupled with an injector, to form an assembly according to aspects of the present disclosure, wherein the assembly is in a first position;

FIG. 3C is view of the assembly shown in FIG. 3B in a second position;

FIG. 3D is a view of the needle shield indicator aid shown in FIG. 3A, coupled with an injector, to form an assembly according to aspects of the present disclosure, wherein the assembly is in a first position and a portion of the indicator aid is translucent for illustrative purposes;

FIG. 3E shows an alternative design for a spring-biased component similar to that shown in FIGS. 3A-3D;

FIGS. 4A-4B are side views of another exemplary drug delivery device assembly according to aspects of the present disclosure, including an accessory configured to indicate to a user whether the needle shield is in the extended position (FIG. 4A) or the retracted position (FIG. 4B);

FIG. 4C is a view of the assembly from FIG. 4A, but with certain components shown in transparent form for illustrative purposes;

FIG. 4D is a cross-sectional view of the assembly from FIG. 4A;

FIGS. 5A-5B are side views of another exemplary drug delivery device assembly according to aspects of the present disclosure, including an accessory configured to indicate to a user whether the needle shield is in the extended position (FIG. 5A) or the retracted position (FIG. 5B);

FIGS. 6A-6B are side views of another exemplary drug delivery device assembly according to aspects of the present disclosure, including an accessory configured to indicate to a user whether the needle shield is in the extended position (FIG. 6A) or the retracted position (FIG. 6B);

FIGS. 7A-7C are side views of another exemplary drug delivery device assembly according to aspects of the present disclosure, including an accessory configured to indicate to a user whether the needle shield is in the extended position (FIG. 7A), the retracted position (FIG. 7B), or an unlocked position (FIG. 7C);

FIGS. 7D-7F are cross-sectional views of the assembly from FIG. 7A;

FIGS. 8A-8B are side views of another exemplary drug delivery device assembly according to aspects of the present disclosure, including an accessory configured to indicate to a user whether the needle shield is in the extended position (FIG. 8A) or the retracted position (FIG. 8B);

FIG. 8C is a cross-sectional view of the assembly from FIG. 8A;

FIGS. 9A-9B are side views of another exemplary drug delivery device assembly according to aspects of the present disclosure, including an accessory configured to indicate to a user whether the needle shield is in the extended position (FIG. 9A) or the retracted position (FIG. 9B);

FIG. 9C is a cross-sectional view of the assembly from FIG. 9A;

FIG. 9D is a view of the assembly from FIG. 9A, but with certain components shown in transparent form for illustrative purposes;

FIGS. 10A-10B are side views of another exemplary drug delivery device assembly according to aspects of the present disclosure, including an accessory configured to indicate to a user whether the needle shield is in the extended position (FIG. 10A) or the retracted position (FIG. 10B);

FIGS. 10C-10D are views of the assembly from FIG. 10A, but with certain components shown in transparent form for illustrative purposes; and

FIGS. 11A-11B are side views of another exemplary drug delivery device assembly according to aspects of the present disclosure, including an accessory configured to indicate to a user whether the needle shield is in the extended position (FIG. 11A) or the retracted position (FIG. 11B).

### DETAILED DESCRIPTION

Generally speaking, pursuant to these various embodiments, a drug delivery device (e.g., an autoinjector or other injector) is coupled with or used in conjunction with an accessory to indicate to a user information regarding the needle shield. For example, the accessory may include an accessory body for selectively coupling the accessory and the injector housing and a needle shield indicator configured to indicate to a user whether the needle shield is in the retracted position.

The term “about” as used herein means +/−10% to the smallest significant digit. The term “patient's skin” may refer to the users uncovered, naked, or bare skin and/or the user's skin as it is covered by clothing, bandage, or other covering.

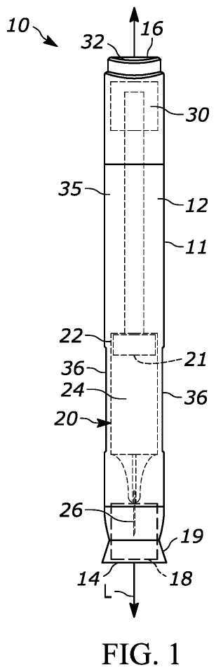

As illustrated in FIG. 1, an example injector 10 generally includes an injector housing 11 defining a housing 12 that includes a distal end 14, a proximal end 16, and a longitudinal axis L extending between the distal and proximal ends 14, 16. The injector 10 distal end 14 includes a generally cylindrical shaped needle shield 18 that assists with actuation of the injector 10 and a needle cap 19 that covers the needle shield 18 prior to use of the injector. A needle assembly 20 is at least partially disposed within the housing 12 at or near the distal end 14, and includes a syringe barrel 22 that contains a medicament 24, a plunger stopper 21 disposed within the syringe barrel 22, and a needle or a cannula 26 that is used to inject the medicament 24 into a patient at a desired injection site. In the illustrated example, the needle or cannula 26 is initially positioned within the housing 12 prior to activation, and may protrude through an opening in the distal end 14 during drug delivery.

A drive assembly 30 is also at least partially disposed within the housing 12 and is operably coupled to the needle assembly 20. The drive assembly 30 may include an actuator button 32 positioned at or near the proximal end 16 of the housing 12 that initiates actuation of the drive assembly 30. In operation, a user removes the needle cap 19, places the needle shield 18 against the injection location (e.g., on their leg or their stomach), and actuates the actuator button 32. This actuation causes a drive mechanism (in the form of a spring, a motor, a hydraulic or pressurized mechanism, etc.) of the drive assembly 30 to exert a driving force on a portion of the needle assembly 20, such as the plunger stopper 21, that causes the needle or cannula 26 to be inserted through the opening of the housing 12 and into a patient and/or that further causes the medicament 24 to be urged from the syringe barrel 22, out the needle or cannula 26, and into the patient. In some versions, the patient may manually insert the needle or cannula 26, and actuation of the drive mechanism 30 only includes urging the plunger stopper 21 in the distal direction thereby causing the medicament 24 to be urged from the syringe barrel 22, out the needle or cannula 26, and to the patient. The injector 10 may not include an actuator button and may instead be activated by movement of the needle shield 18 alone, rather than an actuator button plus movement of the needle shield.

The injector 10 may include any number of additional features and components that may assist and/or enhance the functionality of the device. In the illustrated example, a viewing window 36 positioned at or near the syringe barrel 22 provides a visual indication of the remaining quantity of drug during administration. The needle cap 19 shields the needle 26 and prevents unintentional activation of the injector 10 and deployment of the needle or cannula 26. The needle cap 19 acts to unlock or initiate the injection when the needle shield 18 is pressed against a patient's skin. The activation of the drive assembly 30 requires a specific force to be applied to the needle shield 18 of the injector 10 and that force is transferred to the users skin. In other examples, the injector 10 may additionally include one or more electronic modules that are coupled to the housing 12, the needle assembly 20, the drive assembly 30, and/or any other components of the injector 10. Further, the injector 10 may also include any number of safety mechanisms such as a retraction mechanism, damping mechanism, and the like.

The present example of the drug delivery device 10 takes the form of an autoinjector or pen-type injector, and, as such, may be held in the hand of the user over the duration of drug delivery. The drug delivery device 10 may be suitable for self-administration by a patient or for administration by caregiver or a formally trained healthcare provider (e.g., a doctor or nurse).

However, various implementations and configurations of the drug delivery device 10 are possible. In other examples, the drug delivery device 10 may be configured as a multiple-use reusable injector.

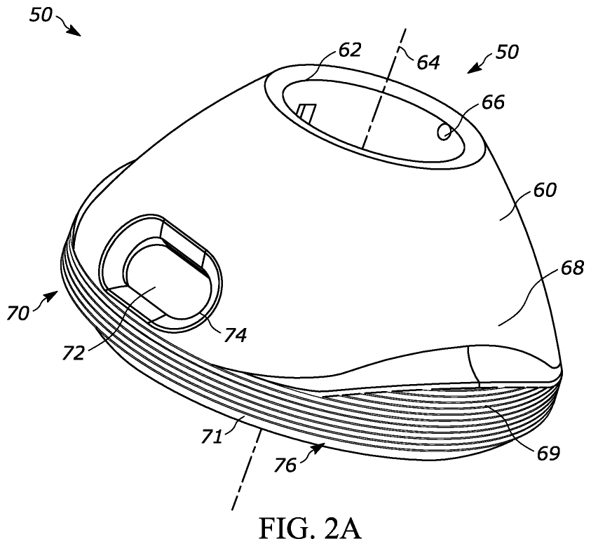

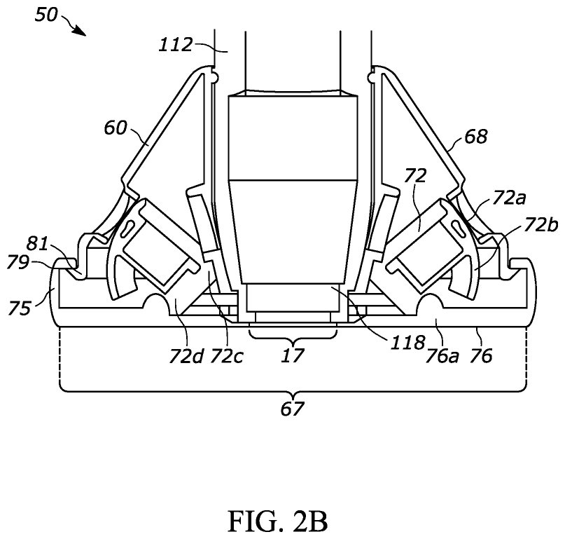

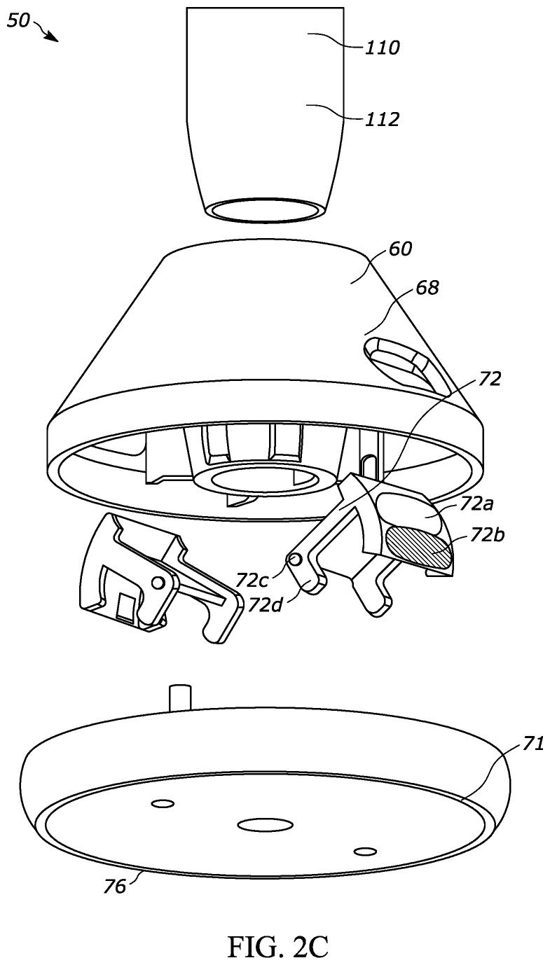

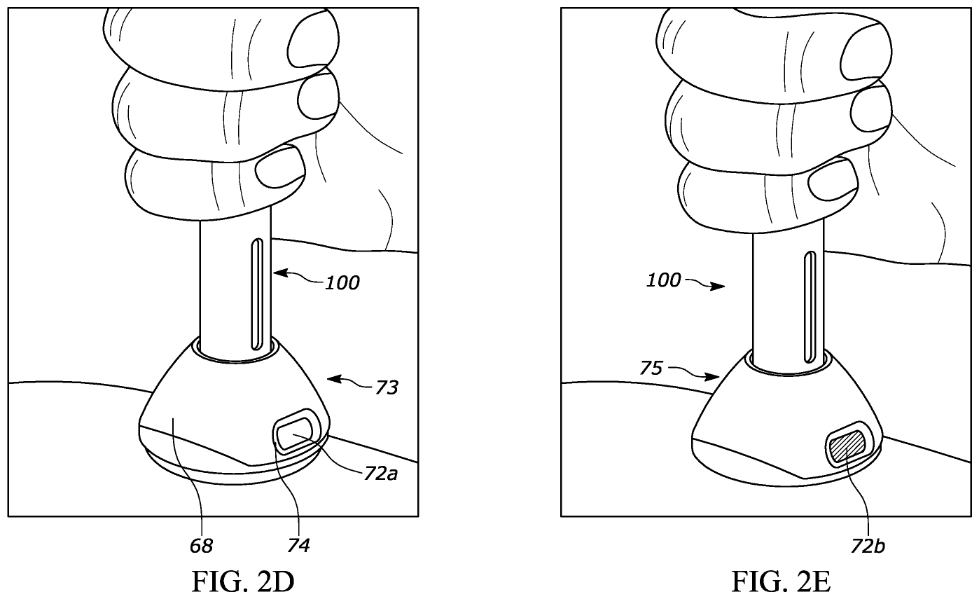

FIGS. 2A-2E show a drug delivery device assembly 100, including an injector 110 and an accessory 50 according to aspects of the present disclosure. The accessory 50 includes an accessory body 60 configured to be selectively coupled with the injector housing 112, a needle shield indicator 70 configured to indicate to a user whether the needle shield is in the retracted position.

As shown in FIG. 2A, the accessory body 60 includes a cylindrical opening 62 near the proximal end of the body 60 and extending substantially or along a longitudinal axis 64. The cylindrical opening 62 may receive the distal portion of the injector housing 112, such as the portion of the injector 110 adjacent to the needle shield 118. The cylindrical opening 62 may have a diameter and shape so as to form a friction fit with the injector 110. Additionally or alternatively, cylindrical opening 62 may define a coupling feature such as a protrusion 66 that extends radially inward (towards the longitudinally axis 64) and enhances or forms the friction fit with the injector 110. As a more specific example, the protrusion 66 may be received within or coupled with a feature of the injector housing 112. As an even more specific example, the protrusion 66 may be received within and/or engage a surface of the drug window 36 to help couple the injector 110 and the accessory 50. The injector 110 may be removed from engagement with the accessory 50 by pulling and/or rotating the injector 110 with respect to the accessory 50. Additionally or alternatively, other coupling features may be suitable. For example, the accessory body 60 may have locking arms that are able to flex radially outward while the injector is being slid into the accessory body and to move radially inward when the injector is in its fully-inserted position.

The accessory body 60 defines a generally bell-shaped housing 68 having a diameter 67 at the distal end that is significantly larger than a diameter 17 of the distal end of injector housing 112. This relatively wide diameter 67 will be discussed in more detail below. The accessory body 60 includes grip ridges 69 that may assist a user in gripping the accessory 50 during handling or operation.

As mentioned above, the accessory 50 includes needle shield indicator 70 configured to indicate to a user whether the needle shield is in the retracted position. The needle shield indicator 70 shown in FIGS. 2A-2E includes a feedback component configured to provide the user feedback when the needle shield is in the retracted position. For example, the feedback component shown in FIGS. 2A-2E generally includes at least one movable indicator 72 that is able to pivot between first and second positions to signify whether the needle shield 118 is in the extended position or the retracted position and/or a movable plate 71 that is movable with respect to the accessory body 60 along the longitudinal axis 64. As a more specific example, the indicator 72 provides the user with a visual indicator when the needle shield 118 is in the retracted position. As shown in FIG. 2C, the indicator 72 has a first viewing surface 72a that is visible when the indicator 72 is in a first position 73 (FIG. 2D) and the needle shield 118 is in the extended position and a second viewing surface 72b that is visible when the indicator is in a second position 75 (FIG. 2E) and the needle shield 118 is in the retracted position.

The movable indicator 72 shown in the figures (particularly FIGS. 2B and 2C) includes a fulcrum and a lever arm. The fulcrum on each movable indicator 72 may be defined by two connector points such as two knobs 72c that are coupled with the accessory body 60 in such a way as to permit and/or promote pivoting movement about an axis extending through the knobs 72c. The coupling relationship between the knobs 72c and the body 60 may be a snap-fit connection, a frictional fit connection, or another suitable relationship that permits the pivoting movement. The lever arm on each movable indicator 72 may be one or more arms 72d that are aligned with a portion of the contact surface 76 such that as the contact surface 76 moves upward (in the proximal direction) relative to the accessory body 60 the arms 72d are urged upward and cause the aforementioned pivoting movement. As an even more specific example, the contact surface 76 includes protrusions 76a aligned with the lever arms 72d such as to urge upward the lever arms 72d when the contact surface 76 moves upward (in the proximal direction) relative to the accessory body 60.

The movable plate 71 shown in the figures is coupled with the housing 68 such as to permit relative movement between the respective components 71, 68 along the longitudinal axis 64. As a more specific example, the movable plate 71 may have a travel distance equal to or substantially equal to the travel distance of the needle shield 118. As an even more specific example, when the movable plate 71 is in a fully extended position (FIGS. 2B and 2D) the needle shield 118 may be in the extended position; and when the movable plate 71 is in a fully retracted position (FIG. 2C) the needle shield 118 may be in the retracted position.

The movable plate 71 may be coupled with the housing 68 in any suitable manner, such as an annular ledge 79 around the outer surface of the movable plate 71 that sits on an annular ledge 81 of the housing 68. The movable plate 71 may be spring-loaded with respect to the housing 68 such that the movable plate 71 is in the extended position unless and until a force is applied in the proximal direction, such as a force by a user pressing the assembly 100 against the users skin at the injection site.

The proximal side of the movable plate 71 shown in the figures includes protrusions 76 for selectively engaging and/or urging the arms 72d in the proximal direction, as discussed above.

The relatively wide diameter of the contact surface 76 provides the user with a wider surface (i.e., wider than the distal portion of the injector 100) for contacting the user's skin, such that the contact forces acting on the user's skin may be more spread out, the user's skin may be less likely to buckle or fold during contact, and/or it may be easier for the user to hold the drug delivery device in the desired orientation (perpendicular to the injection site). As a more specific example, the diameter of the contact surface 76 is approximately 4 to 5 times larger than the diameter of the injector 110. Alternatively, the diameter of the contact surface may be any suitable size, such as 1.5 to 2 times larger than the diameter of the injector, 2 to 3 times larger than the diameter of the injector, 3 to 4 times larger than the diameter of the injector, 4 to 5 times larger than the diameter of the injector, 5 to 6 times larger than the diameter of the injector, or any other suitable size. The size of the contact surface 76 may also serve another function, such as helping the user properly align the injector in a direction such that the needle is generally perpendicular to the patient's skin at the injection site. As a more specific example, some users may find it easier to align a larger contact surface (e.g., the contact surface 76) with the injection site because it is easier to see and/or feel when the contact surface 76 is parallel with and/or pressed flat against the skin. As another example, the contact surface 76 may also flatten the users skin at the injection site, thereby causing a more predictable and/or repeatable injection experience.

During use, a user may hold the injector 110 by the housing 112 and align the assembly 100 near the user's skin at the desired injection state, as shown in FIG. 2D. At this point, the first viewing surface 72a is visible through a window 74 in the housing 68, thereby indicating and/or confirming to the user that the movable plate 71 and the needle shield 118 are in the extended position (e.g., the first position 73). Next, the user may press downward (distally) along the longitudinal axis such that the accessory body 60 moves downward (distally) with respect to the movable plate 71, thereby causing the movable indicators 72 to pivot and display the second viewing surface 72b through the window 74. At the same time, the housing 112 of the injector moves downward (distally) with respect to the needle shield 118, causing the needle shield to move into the retracted position. Note that during this operation, the needle shield 118, the movable plate 71, and the user's skin at the injection site may remain stationary while the injector housing 112 and the accessory body 60 move distally. Alternatively, the user may move his/her skin upward towards the accessory 150, thereby causing the needle shield 118 and the movable plate 71 to move proximally with respect to the housing 112 and the accessory body 60. But in either instance, relative movement occurs between the needle shield 118 and the housing 112 and the movable plate 71 and the body 60. And in either case, when the second viewing surface 72b is visible through the window 74 then the user will be able to receive feedback that the needle shield 118 is retracted and the injection is ready to start or has already started (depending on whether the injector requires an additional step such as pressing an actuation button).

The accessory 50 may include any suitable number of viewing windows 74 such as the two shown in the figures, one window, four windows, 6 windows, 8 windows, or any suitable number of windows. The accessory may further or alternatively include other indicators such as an audible indicator (such as a click or another audible sound) or a haptic indicator (such as a vibration or other movement of the accessory).

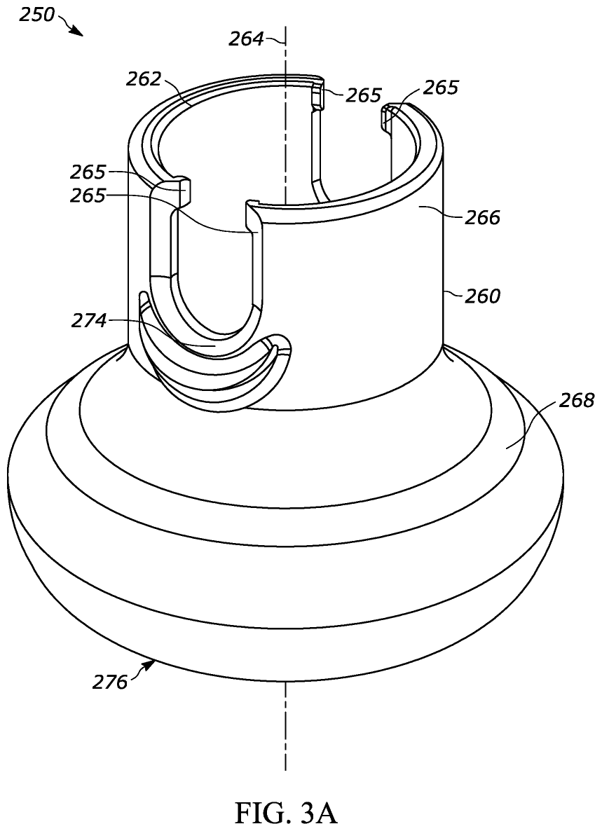

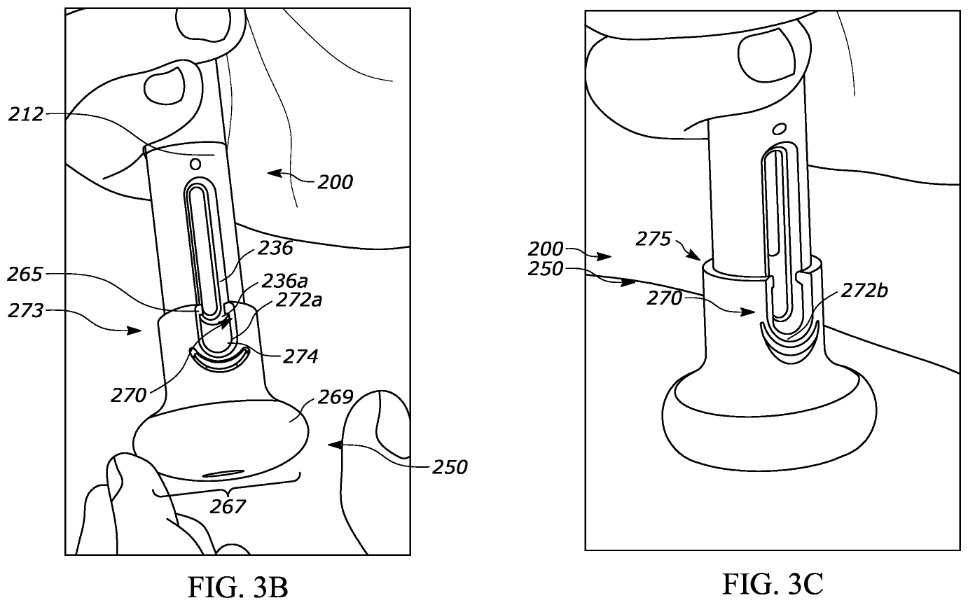

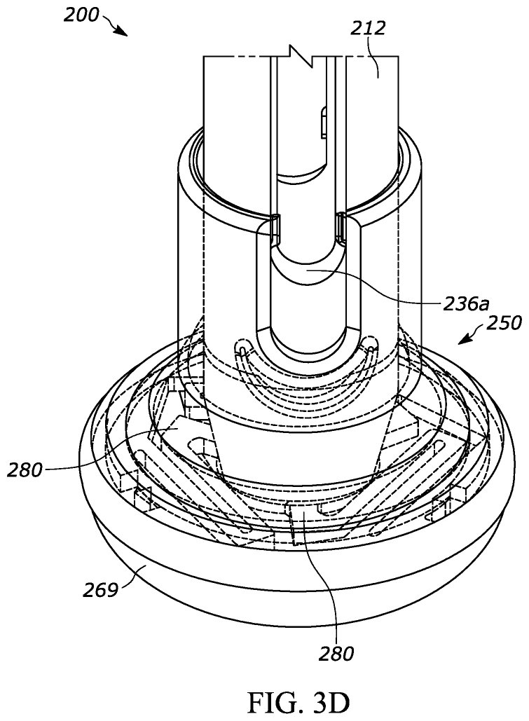

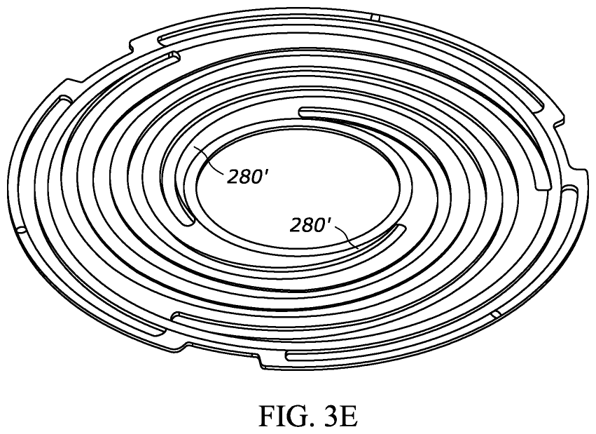

FIGS. 3A-3D show a drug delivery device assembly 200, including an injector 210 and an accessory 250 according to aspects of the present disclosure. The accessory 250 includes an accessory body 260 configured to be selectively coupled with the injector housing 212, a needle shield indicator 270 configured to indicate to a user whether the needle shield is in the retracted position.

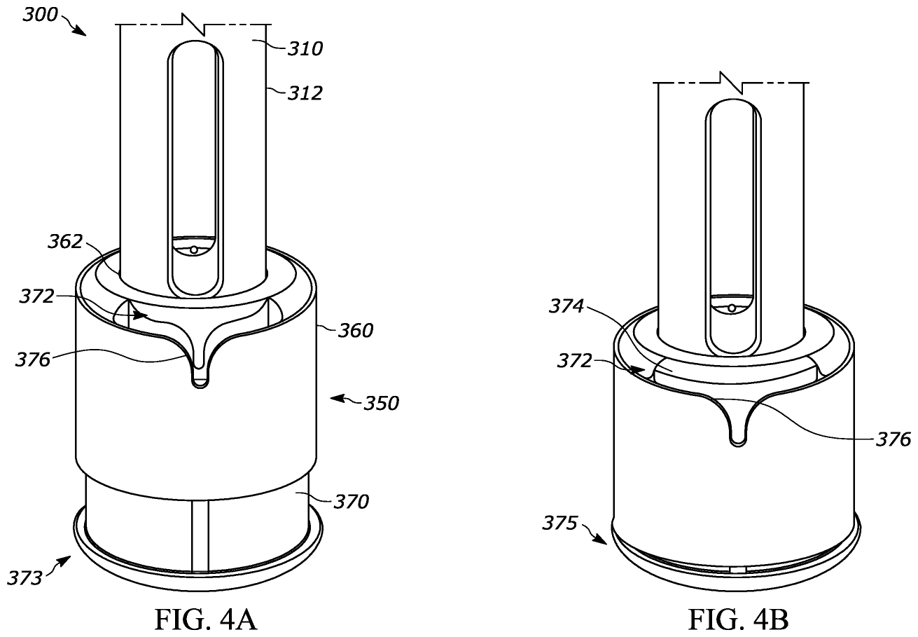

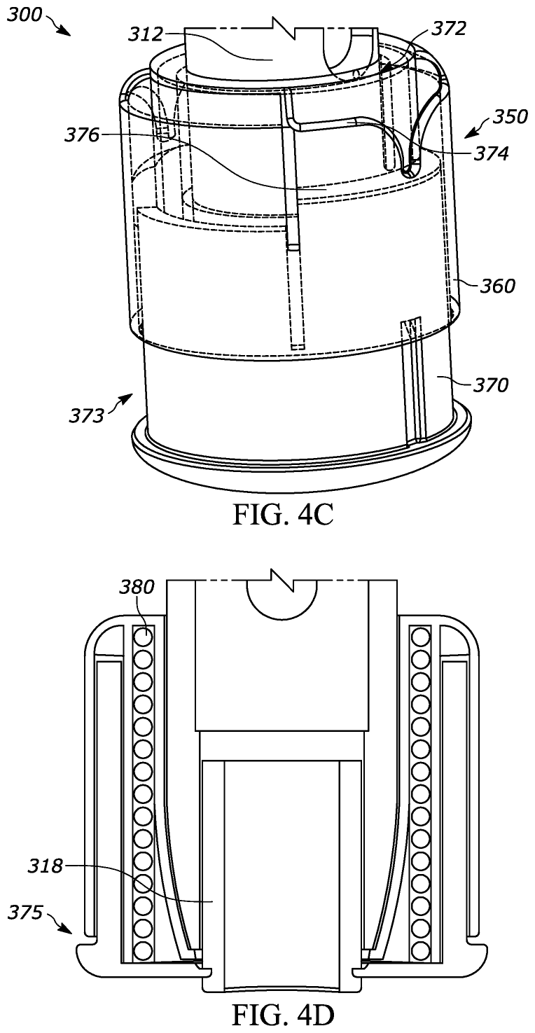

As shown in FIG. 3A, the accessory body 260 includes a cylindrical opening 262 near the proximal end of the body 260 and extending substantially or along a longitudinal axis 264. The cylindrical opening 262 may receive the distal portion of the injector housing 212, such as the portion of the injector 210 adjacent to the needle shield. The cylindrical opening 262 may have a diameter and shape so as to form a friction fit with the injector 210. Additionally or alternatively, cylindrical opening 262 may define a coupling feature such as a protrusion that extends radially inward and enhances or forms the friction fit with the injector 210. The injector 210 may be removed from engagement with the accessory 250 by pulling and/or rotating the injector 210 with respect to the accessory 250. Additionally or alternatively, other coupling features may be suitable. For example, the accessory body 260 may have locking arms that are able to flex radially outward while the injector is being slid into the accessory body and to move radially inward when the injector is in its fully inserted position. As a more specific example, as shown in FIG. 4D, the accessory 250 may include spring-biased arms 280 that are able to flex outward to receive the injector housing 212 but also to stay biased inwardly to form a frictional fit between the accessory 250 and the injector 210. The spring-biased arms 280 may also be configured to apply an upward force on the injector housing 212 to hold the injector 210 in place with respect to the accessory 250. Additionally, the accessory may include securing tabs 265 that engage with the injector window 236 to counteract the upward force of the spring-biased arms 280 and hold the injector 210 in place with respect to the accessory 250. In other words, the spring-biased arms 280 and the securing tabs 265 cooperate to form an equilibrium between the injector 210 and the accessory 250. After injection is complete, the user may also be able to de-couple the injector 210 to be removed from the accessory 250 by pressing down further on the spring-biased arms 280 and causing the spring force of the spring-biased arms 280 to urge the injector 210 out of the accessory 250.

The accessory body 260 defines a generally bell-shaped housing 268 having a diameter 267 at the distal end that is significantly larger than a diameter of the distal end of injector housing. This relatively wide diameter 267 will be discussed in more detail below. The accessory body 260 includes a gripping surface 269 such as a rubber overmold that may grip to the users skin during the injection and help flatten or stretch the skin and/or may assist a user in gripping the accessory 250 during handling or operation.

As mentioned above, the accessory 250 includes needle shield indicator 270 configured to indicate to a user whether the needle shield is in the retracted position. The needle shield indicator 270 shown in FIGS. 3A-3D includes a feedback component configured to provide the user feedback when the needle shield is in the retracted position. For example, the feedback component shown in FIGS. 3A-3D generally includes accessory reference feature 274, wherein the injector housing 212 includes an injector reference feature, such as a drug viewing window 236. When the accessory reference feature 274 and the drug viewing window 236 are aligned when the needle shield is in the retracted position. As a more specific example, in FIG. 3B, the injector housing 212 is in a first position 273 with respect to the accessory 250 such that the needle shield is extended (i.e., the needle shield is not being retracted by the accessory 250 or the users skin at the injection site). At this point, the user is able to observe a gap or space between the accessory reference feature 274 and the drug viewing window 236 to determine or confirm that the injector housing 212 is in the first position 273. Also, in FIG. 3C, the injector housing 212 is in a second position 275 with respect to the accessory 250 such that the needle shield is retracted (i.e., the needle shield has been depressed by the accessory 250 and/or the users skin at the injection site). At this point, the user is able to observe that there is no gap between the accessory reference feature 274 and the drug viewing window 236 (i.e., the features 274, 236 are aligned) to determine or confirm that the injector housing 212 is in the second position 273.

The accessory reference feature 274 and the drug viewing window 236 may have similar shapes to emphasize or highlight the alignment features, such as the general arcuate shape shown in FIGS. 3B and 3C, but any other suitable shapes may be used. The accessory reference feature 274 and the drug viewing window 236 may also be configured to mate with each other, such as a arcuate rib 236a at the distal end of the drug viewing window 236 shown in FIG. 3B that presses up against the accessory reference feature 274 when the injector housing 212 is in the second position 275.

The accessory 250 includes a contact surface 276 having a diameter that is wider than the diameter of the distal end of the injector 200. The relatively wide diameter of the contact surface 276 provides the user with a wider surface (i.e., wider than the distal portion of the injector 200) for contacting the user's skin, such that the contact forces acting on the user's skin may be more spread out, the users skin may be less likely to buckle or fold during contact, and/or it may be easier for the user to hold the drug delivery device in the desired orientation (perpendicular to the injection site). As a more specific example, the diameter of the contact surface 276 is approximately 4 to 5 times larger than the diameter of the injector 210. Alternatively, the diameter of the contact surface may be any suitable size, such as 1.5 to 2 times larger than the diameter of the injector, 2 to 3 times larger than the diameter of the injector, 3 to 4 times larger than the diameter of the injector, 4 to 5 times larger than the diameter of the injector, 5 to 6 times larger than the diameter of the injector, or any other suitable size. The size of the contact surface 276 may also serve another function, such as helping the user properly align the injector in a direction such that the needle is generally perpendicular to the patient's skin at the injection site. As a more specific example, some users may find it easier to align a larger contact surface (e.g., the contact surface 276) with the injection site because it is easier to see and/or feel when the contact surface 276 is parallel with and/or pressed flat against the skin. As another example, the contact surface 276 may also flatten the user's skin at the injection site, thereby causing a more predictable and/or repeatable injection experience.

During use, a user may hold the injector 210 by the housing 212 and insert the injector 210 into the accessory 250 in the first position 273, as shown in FIG. 3B. At this point, the accessory reference feature 274 and the drug viewing window 236 are spaced apart from each other, thereby indicating and/or confirming to the user that the needle shield is in the extended position (e.g., the first position 273). Next, the user may place the contact surface 276 against the user's skin at the injection site and press downward (distally) along the longitudinal axis such that the housing 212 moves downward (distally) with respect to the movable plate body 260, thereby causing the accessory reference feature 274 and the drug viewing window 236 to become aligned and/or contacting and/or mating each other. Alternatively, the user may move his/her skin upward towards the accessory 250, thereby causing the accessory 250 and the needle shield to move proximally with respect to the housing 212. But in either instance, relative movement occurs between the housing 212 on one hand and the needle shield and the and the accessory 260 on the other hand. And in either case, when the accessory reference feature 274 and the drug viewing window are in the second position such that the user will be able to receive feedback that the needle shield is retracted and the injection is ready to start or has already started (depending on whether the injector requires an additional step such as pressing an actuation button).

FIG. 3E shows an alternative design for a spring-biased component 280′ that may be utilized in the accessory 250 shown in FIGS. 3A-3D, similar to the spring-biased arms 280 shown in those figures. For example, the spring-biased component 280′ and the securing tabs 265 cooperate to form an equilibrium between the injector 210 and the accessory 250. After injection is complete, the user may also be able to de-couple the injector 210 to be removed from the accessory 250 by pressing down further on the spring-biased arms 280′ and causing the spring force of the spring-biased arms 280′ to urge the injector 210 out of the accessory 250.

FIGS. 4A-4D show a drug delivery device assembly 300, including an injector 310 and an accessory 350 according to aspects of the present disclosure. The accessory 350 includes an accessory body 360 configured to be selectively coupled with the injector housing 312, a needle shield indicator 370 configured to indicate to a user whether the needle shield is in the retracted position.

As shown in FIG. 4A, the accessory body 360 includes a cylindrical opening 362 near the proximal end of the body 360 and extending substantially or along a longitudinal axis. The cylindrical opening 362 may receive the distal portion of the injector housing 312, such as the portion of the injector 310 adjacent to the needle shield. The cylindrical opening 362 may have a diameter and shape so as to form a friction fit with the injector 410.

As mentioned above, the accessory 350 includes needle shield indicator 370 configured to indicate to a user whether the needle shield is in the retracted position. The needle shield indicator 370 may be a different color than the injector and/or the accessory body to make the needle shield indicator 370 more visible to the user. For example, in some versions the needle shield indicator 370 has a bright yellow color. The needle shield indicator 370 shown in FIGS. 4A-4D includes a feedback component 372 configured to provide the user feedback when the needle shield is in the retracted position. For example, the feedback component 372 shown in FIGS. 4A-4D generally includes accessory reference feature 374 and a feedback window 376 that allows a user to view the accessory reference feature 374 when the needle shield is in the retracted position. As a more specific example, in FIG. 4A, the needle shield indicator 370 is in a first position 373 with respect to the accessory body 360 to indicate that the needle shield is extended (i.e., the needle shield is not being retracted by the accessory 350 or the users skin at the injection site). At this point, the user is not able to observe the accessory reference feature 374 in the feedback window 376, indicating that the shield indicator 370 (and the needle shield itself) are in the extended position. Conversely, in FIG. 4B, the needle shield indicator 370 is in a second position 375 with respect to the accessory body 360 to indicate that the needle shield is retracted (i.e., the needle shield is being retracted by the accessory 350 or the user's skin at the injection site). At this point, the user is able to observe the accessory reference feature 374 in the feedback window 376, indicating that the shield indicator 370 (and the needle shield itself) are in the retraced position. When the user removes or releases the downward force on the injector 310, then the needle shield indicator 370 may move back to the first position 373 due to a biasing force from a spring 380 (FIG. 4D).

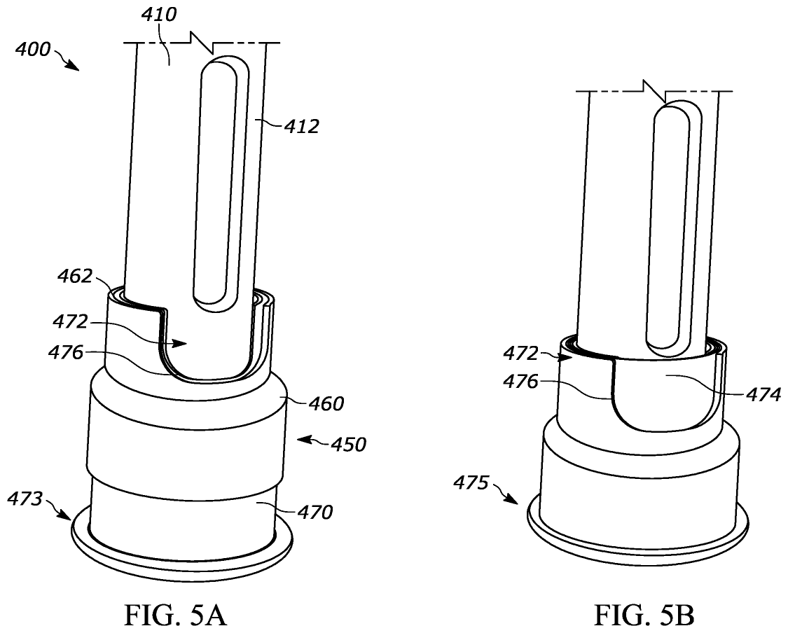

FIGS. 5A-5B show a drug delivery device assembly 400, including an injector 410 and an accessory 450 according to aspects of the present disclosure. The accessory 450 includes an accessory body 460 configured to be selectively coupled with the injector housing 412, a needle shield indicator 470 configured to indicate to a user whether the needle shield is in the retracted position.

As shown in FIG. 5A, the accessory body 460 includes a cylindrical opening 462 near the proximal end of the body 460 and extending substantially or along a longitudinal axis. The cylindrical opening 462 may receive the distal portion of the injector housing 412, such as the portion of the injector 410 adjacent to the needle shield. The cylindrical opening 462 may have a diameter and shape so as to form a friction fit with the injector 410.

As mentioned above, the accessory 450 includes needle shield indicator 470 configured to indicate to a user whether the needle shield is in the retracted position. The needle shield indicator 470 may be a different color than the injector and/or the accessory body to make the needle shield indicator 470 more visible to the user. For example, in some versions the needle shield indicator 470 has a bright yellow color. The needle shield indicator 470 shown in FIGS. 5A-5B includes a feedback component 472 configured to provide the user feedback when the needle shield is in the retracted position. For example, the feedback component 472 shown in FIGS. 5A-5B generally includes accessory reference feature 474 and a feedback window 476 that allows a user to view the accessory reference feature 474 when the needle shield is in the retracted position. As a more specific example, in FIG. 5A, the needle shield indicator 470 is in a first position 473 with respect to the accessory body 460 to indicate that the needle shield is extended (i.e., the needle shield is not being retracted by the accessory 450 or the users skin at the injection site). At this point, the user is not able to observe the accessory reference feature 474 in the feedback window 476, indicating that the shield indicator 470 (and the needle shield itself) are in the extended position. Conversely, in FIG. 5B, the needle shield indicator 470 is in a second position 475 with respect to the accessory body 460 to indicate that the needle shield is retracted (i.e., the needle shield is being retracted by the accessory 450 or the user's skin at the injection site). At this point, the user is able to observe the accessory reference feature 474 in the feedback window 476, indicating that the shield indicator 470 (and the needle shield itself) are in the retraced position. When the user removes or releases the downward force on the injector 410, then the needle shield indicator 370 may move back to the first position 373 due to a biasing force from a spring (not shown).

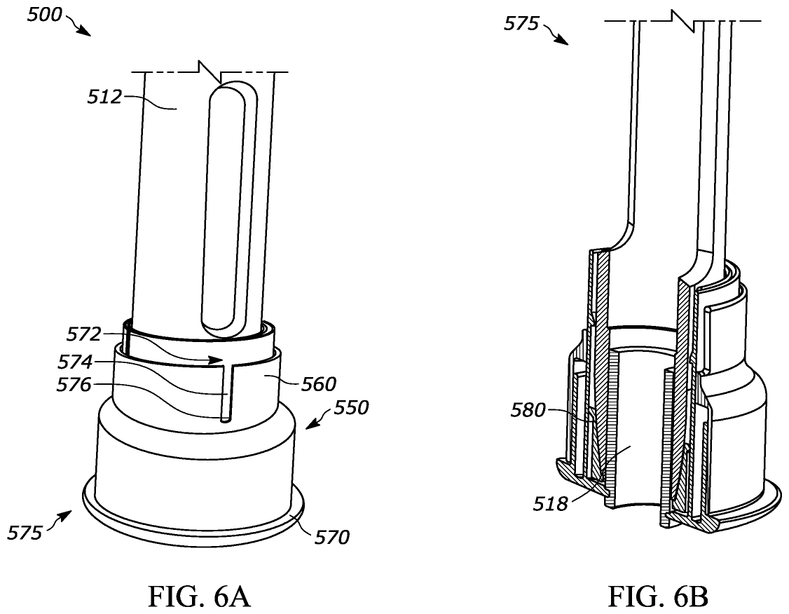

FIGS. 6A-6B show a drug delivery device assembly 500, including an injector 510 and an accessory 550 according to aspects of the present disclosure. The accessory 550 includes an accessory body 560 configured to be selectively coupled with the injector housing 512, a needle shield indicator 570 configured to indicate to a user whether the needle shield is in the retracted position.

As shown in FIG. 6A, the accessory body 560 includes a cylindrical opening 562 near the proximal end of the body 560 and extending substantially or along a longitudinal axis. The cylindrical opening 562 may receive the distal portion of the injector housing 512, such as the portion of the injector 510 adjacent to the needle shield. The cylindrical opening 562 may have a diameter and shape so as to form a friction fit with the injector 510.

As mentioned above, the accessory 550 includes needle shield indicator 570 configured to indicate to a user whether the needle shield is in the retracted position. The needle shield indicator 570 may be a different color than the injector and/or the accessory body to make the needle shield indicator 570 more visible to the user. For example, in some versions the needle shield indicator 570 has a bright yellow color. The needle shield indicator 570 shown in FIGS. 6A-6B includes a feedback component 572 configured to provide the user feedback when the needle shield is in the retracted position. For example, the feedback component 572 shown in FIGS. 6A-6B generally includes accessory reference feature 574 and a feedback window 576 that allows a user to view the accessory reference feature 574 when the needle shield is in the retracted position. As a more specific example, the needle shield indicator 570 may be in a first position (similar to FIG. 5A) with respect to the accessory body 560 to indicate that the needle shield is extended (i.e., the needle shield is not being retracted by the accessory 550 or the users skin at the injection site). At this point, the user is not able to observe the accessory reference feature 574 in the feedback window 576, indicating that the shield indicator 570 (and the needle shield itself) are in the extended position. Conversely, in FIG. 6A, the needle shield indicator 570 is in a second position 575 with respect to the accessory body 560 to indicate that the needle shield is retracted (i.e., the needle shield is being retracted by the accessory 550 or the users skin at the injection site). At this point, the user is able to observe the accessory reference feature 574 in the feedback window 576, indicating that the shield indicator 570 (and the needle shield itself) are in the retraced position. When the user removes or releases the downward force on the injector 510, then the needle shield indicator 570 may move back to the first position 573 due to a biasing force from a spring (not shown). The accessory 550 may also include spring arms 580 that are biased outward or inward and are able to move outward or inward once the shield indicator 570 is in the retracted position 575. The spring arms 580 may provide an audible or tactile indicator to the user, thereby indicating that the shield indicator 570 is in the retracted position 575.

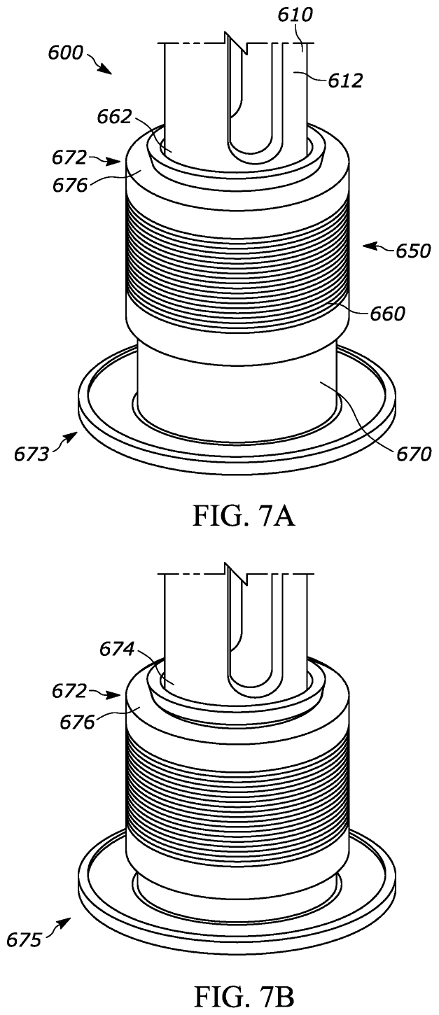

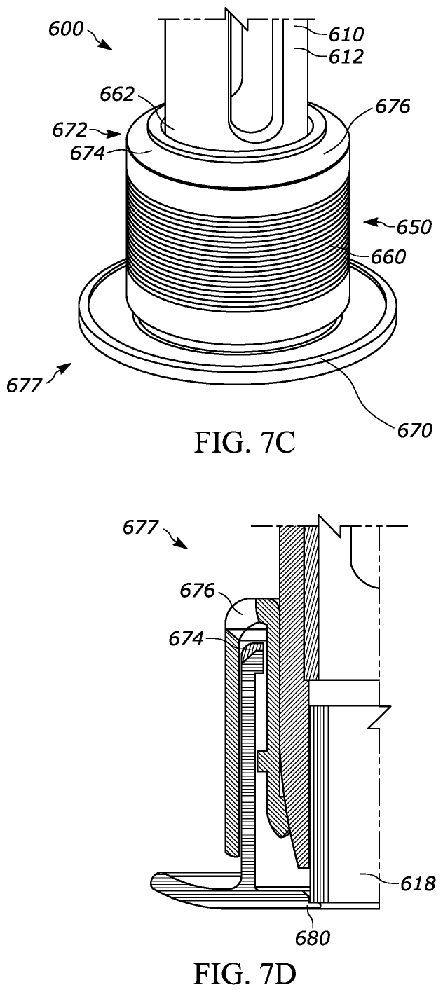

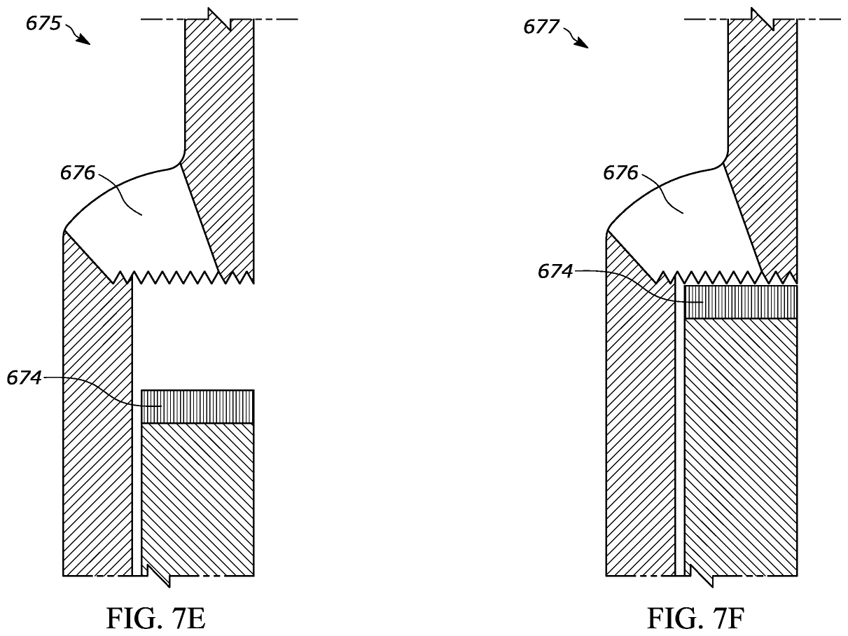

FIGS. 7A-7F show a drug delivery device assembly 600, including an injector 610 and an accessory 650 according to aspects of the present disclosure. The accessory 650 includes an accessory body 660 configured to be selectively coupled with the injector housing 612, a needle shield indicator 670 configured to indicate to a user whether the needle shield is in the retracted position.

As shown in FIG. 7A, the accessory body 660 includes a cylindrical opening 662 near the proximal end of the body 660 and extending substantially or along a longitudinal axis. The cylindrical opening 662 may receive the distal portion of the injector housing 612, such as the portion of the injector 610 adjacent to the needle shield. The cylindrical opening 662 may have a diameter and shape so as to form a friction fit with the injector 610.

As mentioned above, the accessory 650 includes needle shield indicator 670 configured to indicate to a user whether the needle shield is in the retracted position. The needle shield indicator 670 shown in FIGS. 7A-7F includes a feedback component 672 configured to provide the user feedback when the needle shield is in the retracted position. For example, the feedback component 672 shown in FIGS. 7A-7F generally includes accessory reference feature 674 and a feedback window 676 that allows a user to view the accessory reference feature 674 when the needle shield is in the retracted position. The feedback window 676 may be an annular ring that is a transparent or translucent section of the accessory 650. As shown in FIG. 7A, the needle shield indicator 670 is in a first position 673 with respect to the accessory body 660 to indicate that the needle shield is extended (i.e., the needle shield is not being retracted by the accessory 650 or the user's skin at the injection site). At this point, the user is not able to observe the accessory reference feature 674 in the feedback window 676, indicating that the shield indicator 670 (and the needle shield itself) are in the extended position. Conversely, in FIG. 7B and FIG. 7E, the needle shield indicator 670 is in a second position 675 with respect to the accessory body 660 to indicate that the needle shield is retracted (i.e., the needle shield is being retracted by the accessory 650 or the user's skin at the injection site). At this point, the user is able to observe the accessory reference feature 674 in the feedback window 676, indicating that the shield indicator 670 (and the needle shield itself) are in the retraced position. The needle shield indicator 670 may be a different color than the injector and/or the accessory body to make the needle shield indicator 670 more visible to the user. For example, in some versions the needle shield indicator 670 has a bright yellow color. In FIG. 7C, FIG. 7D, and FIG. 7F, the needle shield indicator 670 is in a third position 677 with respect to the accessory body 660 to indicate that the injector 600 is unlocked from the accessory 650 (i.e., the injector can be removed from the accessory 650). For example, when the needle shield indicator 670 is in the third position 677, a bottom flange portion 680 impacts the needle shield 618 and urges the needle shield 618 upward and out of the accessory, thereby making it easier to remove the injector 600 from the accessory 650 after the injection is completed. As an additional or alternative design, the injector may be urged into the third position 677 by a spring release design with a camming/unlocking surface.

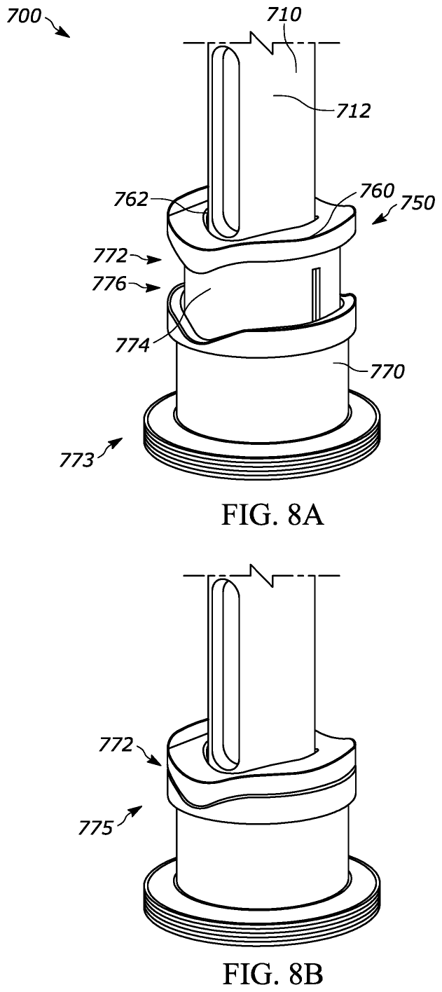

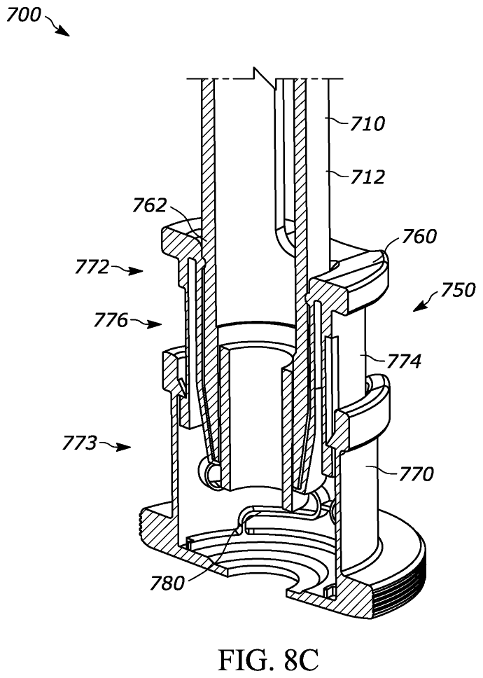

FIGS. 8A-8B show a drug delivery device assembly 700, including an injector 710 and an accessory 750 according to aspects of the present disclosure. The accessory 750 includes an accessory body 760 configured to be selectively coupled with the injector housing 712, a needle shield indicator 770 configured to indicate to a user whether the needle shield is in the retracted position.

As shown in FIG. 8A, the accessory body 760 includes a cylindrical opening 762 near the proximal end of the body 760 and extending substantially or along a longitudinal axis. The cylindrical opening 762 may receive the distal portion of the injector housing 712, such as the portion of the injector 710 adjacent to the needle shield. The cylindrical opening 762 may have a diameter and shape so as to form a friction fit with the injector 710.

As mentioned above, the accessory 750 includes needle shield indicator 770 configured to indicate to a user whether the needle shield is in the retracted position. The needle shield indicator 770 may be a different color than the injector and/or the accessory body to make the needle shield indicator 770 more visible to the user. For example, in some versions the needle shield indicator 770 has a bright yellow color. The needle shield indicator 770 shown in FIGS. 8A-8B includes a feedback component 772 configured to provide the user feedback when the needle shield is in the retracted position. For example, the feedback component 772 shown in FIGS. 8A-8C generally includes accessory reference feature 774 and a feedback window 776 that allows a user to view the accessory reference feature 774 when the needle shield is in the retracted position. As a more specific example, in FIGS. 8A and 8C, the needle shield indicator 770 may be in a first position 773 with respect to the accessory body 760 to indicate that the needle shield is extended (i.e., the needle shield is not being retracted by the accessory 750 or the users skin at the injection site). At this point, the user is able to observe the accessory reference feature 774 in the feedback window 776, indicating that the shield indicator 770 (and the needle shield itself) are in the extended position. Conversely, in FIG. 8B, the needle shield indicator 770 is in a second position 775 with respect to the accessory body 760 to indicate that the needle shield is retracted (i.e., the needle shield is being retracted by the accessory 750 or the users skin at the injection site). At this point, the user is not able to observe the accessory reference feature 774 in the feedback window 776, indicating that the shield indicator 770 (and the needle shield itself) are in the retraced position 775. When the user removes or releases the downward force on the injector 710, then the needle shield indicator 770 may move back to the first position 773 due to a biasing force from a spring 780 (FIG. 8C). For example, the spring 780 may be a plastic, winding arm that is able to compress or expand to bias the shield indicator 770 towards the extended position 773 and/or to urge the injector out of engagement with the accessory 750 after injection. The surfaces of the accessory body 760 and the needle shield indicator 770 that selectively contact each other when the needle shield indicator is in the retracted position 775 may provide an audible or tactile indicator to the user, thereby indicating that the shield indicator 770 is in the retracted position 775.

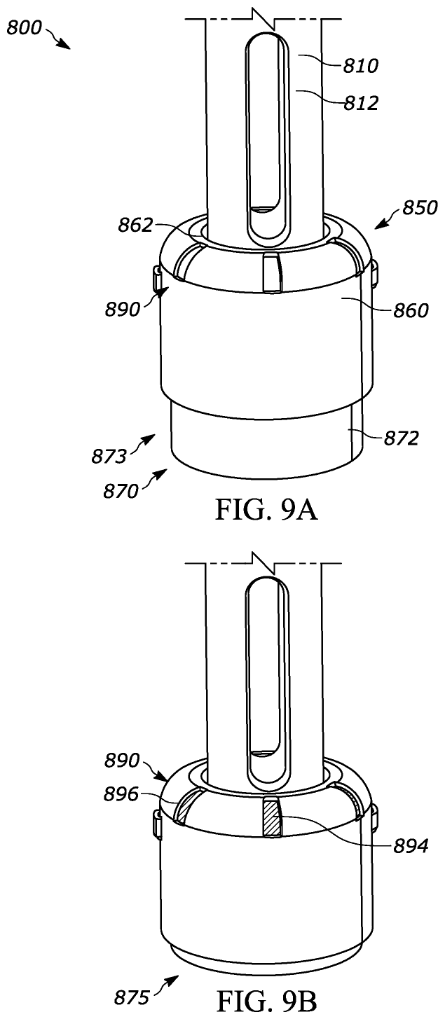

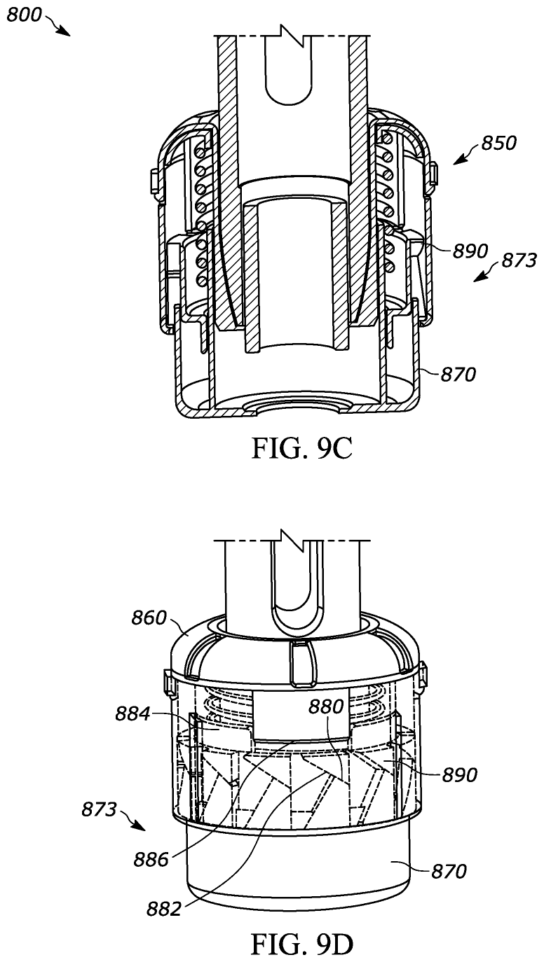

FIGS. 9A-9D show a drug delivery device assembly 800, including an injector 810 and an accessory 850 according to aspects of the present disclosure. The accessory 850 includes an accessory body 860 configured to be selectively coupled with the injector housing 812, a needle shield indicator 870 configured to indicate to a user whether the needle shield is in the retracted position.

As shown in FIG. 9A, the accessory body 860 includes a cylindrical opening 862 near the proximal end of the body 860 and extending substantially or along a longitudinal axis. The cylindrical opening 862 may receive the distal portion of the injector housing 812, such as the portion of the injector 810 adjacent to the needle shield. The cylindrical opening 862 may have a diameter and shape so as to form a friction fit with the injector 810.

As mentioned above, the accessory 850 includes a first needle shield indicator 870 configured to indicate to a user whether the needle shield is in the retracted position. The accessory 850 shown in FIGS. 9A-9D also includes a second needle shield indicator 890. One or both of the needle shield indicators 870, 890 may be a different color than the injector and/or the accessory body to make the needle shield indicators 870, 890 are more visible to the user. For example, in some versions the first needle shield indicator 870 has a bright yellow color and the second needle shield indicator 890 has a bright green color. The needle shield indicators 870, 890 shown in FIGS. 9A-9B each include a feedback component configured to provide the user feedback when the needle shield is in the retracted position. For example, the first needle shield indicator 870 includes a first feedback component 872 that is visible to a user when the needle shield is in the extended position 873 (as in FIG. 9A) but is less visible to a user or not visible to a user when the needle shield is in the retracted position 875 (as in FIG. 9B). The second needle shield indicator 890 includes a second feedback component 892 that is not visible to a user when the needle shield is in the extended position 873 (as in FIG. 9A) but is visible to a user when the needle shield is in the retracted position 875 (as in FIG. 9B).

As shown in FIGS. 9C and 9D, both of which show the needle shield in the extended position 873, the accessory 875 may include camming features and a lockout feature. As a more specific example, the first needle shield indicator 870 includes a camming surface 880 that interfaces with a camming surface 882 on the second needle shield indicator 890 to cause rotational movement of the second needle shield indicator 890 when the first needle shield indicator 870 is urged upward toward the retracted position. When the second needle shield indicator 890 rotates then a lock-out feature on the second needle shield indicator 890 becomes aligned with a lock-out feature 886 on the accessory body 860 to maintain the first needle shield indicator 870 in the extended position. This action may serve as a needle guard and/or to release engagement between the injector and the accessory 850.

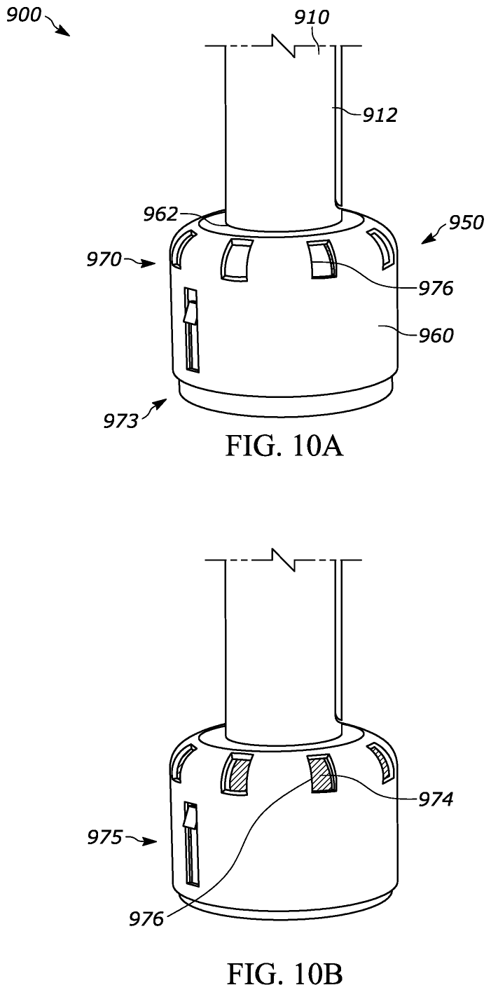

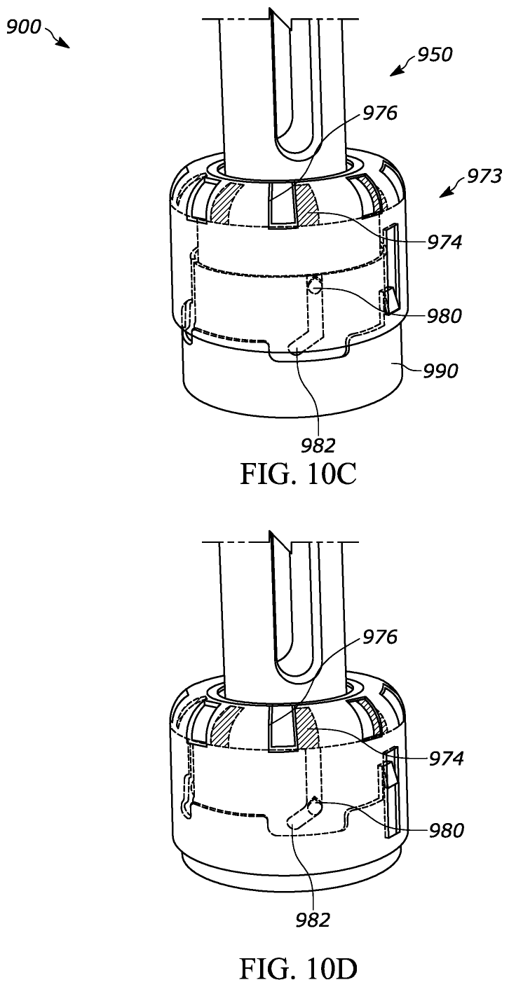

FIGS. 10A-10D show a drug delivery device assembly 900, including an injector 910 and an accessory 950 according to aspects of the present disclosure. The accessory 950 includes an accessory body 960 configured to be selectively coupled with the injector housing 912, a needle shield indicator 970 configured to indicate to a user whether the needle shield is in the retracted position.

As shown in FIG. 10A, the accessory body 960 includes a cylindrical opening 962 near the proximal end of the body 960 and extending substantially or along a longitudinal axis. The cylindrical opening 962 may receive the distal portion of the injector housing 912, such as the portion of the injector 910 adjacent to the needle shield. The cylindrical opening 962 may have a diameter and shape so as to form a friction fit with the injector 910.

As mentioned above, the accessory 950 includes a needle shield indicator 970 configured to indicate to a user whether the needle shield is in the retracted position. The needle shield indicator 970 may be a different color than the injector and/or the accessory body to make the needle shield indicator 970 more visible to the user. For example, in some versions the needle shield indicator 970 has a bright yellow green color. The needle shield indicator 970 shown in FIGS. 10A-10D includes a feedback component configured to provide the user feedback when the needle shield is in the retracted position. For example, the needle shield indicator 970 includes a feedback component 974 that is not visible to a user when the needle shield is in the extended position 973 (as in FIG. 10A) but is visible to a user when the needle shield is in the retracted position 975 (as in FIG. 10B). As a more specific example, shown in FIGS. 10C and 10D where the accessory body 960 is transparent for illustrative purposes, the feedback component 974 includes a plurality of green rectangles that are either not aligned with a plurality of viewing windows 976 (as in the extended position 973 shown in FIGS. 10A, 10C) or are aligned with the viewing windows 976 (as in the retracted position 975 shown in FIG. 10B).

The accessory 950 includes a lower housing 990 that is able to translate upwards with respect to the accessory body 960 as the needle shield moves from the extended position to the retracted position. As this relative movement occurs, the feedback component 974 is also able to rotate to align the green rectangles with the viewing windows 976. As a more specific example, as shown in FIGS. 10C and 10D, the lower housing 990 includes a slot 982 that has a vertical portion and an angled portion. The slot receives a knob 980 of the needle shield indicator 970 such as to urge rotation of the needle shield indicator 970 when the knob 980 moves into the angled portion of the slot 982, thereby aligning the green rectangles with the viewing windows 976. In other words, in FIG. 10D the needle shield is moving from the extended position to the retracted position and the needle shield indicator 970 is about to rotate with respect to the accessory body 960.

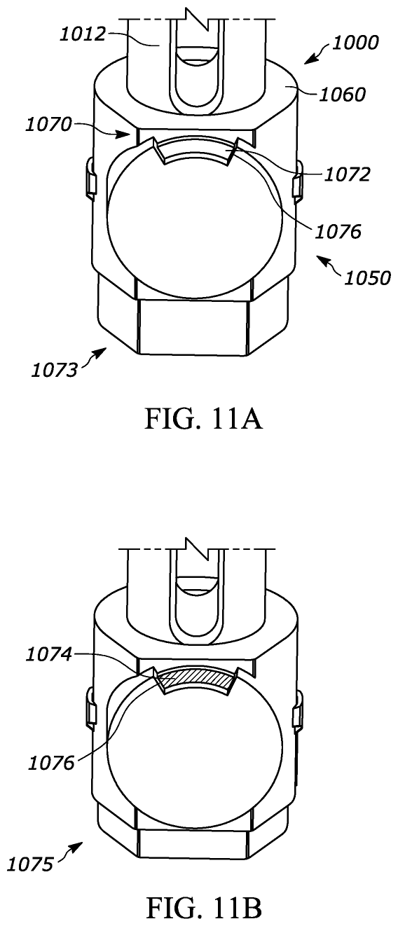

FIGS. 11A-11B show a drug delivery device assembly 1000, including an injector and an accessory 1050 according to aspects of the present disclosure. The accessory 1050 includes an accessory body 1060 configured to be selectively coupled with the injector housing 1012, a needle shield indicator 1070 configured to indicate to a user whether the needle shield is in the retracted position.

As shown in FIG. 11A, the needle shield indicator 1070 includes a feedback component 1072 and a window 1076. The feedback component may be a rotating dial that has a non-colored portion and a colored portion 1074. When the injector needle shield is in an extended position 1073 then the non-colored portion is aligned with the window 1076 and thereby visible to the user. When the injector needle shield is in a retracted position 1075 then the colored portion 1074 is aligned with the window 1076 and thereby visible to the user.

The syringe barrel may have a length of 45 to 85 mm, 60 to 65 mm, or another suitable length. The length of the syringe barrel is the length between the rear end to the outlet to which the needle is attached (but not including the needle, if present).

The syringe barrel may have an internal diameter of 4 to 6.5 mm. If the syringe has a nominal maximum fill volume of 1 ml, the internal diameter of the syringe barrel may be 5.5 to 6.5 mm. If the syringe has a nominal maximum fill volume of 0.5 ml, the internal diameter of the syringe barrel may be 4 to 5 mm.

The wall of the syringe barrel may have a thickness of at least 1 mm; about 1 to 3 mm; about 1.5 to 3 mm; or about 2.4 to 2.8 mm. Due to the thickness of the wall, the sterilizing gas is restricted or prevented from entering interior of the syringe, thereby minimizing or preventing contact with the liquid formulation contained within the prefilled syringe.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RAN KL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIIa receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™ Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG8 mAb (MDX-1307); anti-mesothelin dsFv-PE38 conjugate (CAT-5001); anti-PD1mAb (MDX-1106 (ONO-4538)); anti-PDGFRa antibody (IMC-3G3); anti-TGFR mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-14(R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3× epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device assembly comprising:
an injector housing having a body with a proximal end, a distal end, a longitudinal axis extending between the proximal end and the distal end, and a needle shield positioned adjacent to the distal end and movable between an extended position and a retracted position;
a needle assembly at least partially disposed within the body, the needle assembly comprising a syringe barrel containing a medicament and a needle or a cannula;
a drive assembly at least partially disposed within the body and operably coupled with the needle assembly to urge the medicament through the needle or cannula during an injection sequence; and
a needle shield indicator accessory, including:
an accessory body configured to be selectively coupled with the injector housing; and
a needle shield indicator configured to indicate to a user whether the needle shield is in the retracted position.

2. The drug delivery device assembly as in claim 1, wherein the needle shield indicator includes a feedback component configured to provide the user feedback when the needle shield is in the retracted position.

3. The drug delivery device assembly as in claim 2, wherein the feedback component is movable between a first position and a second position.

4. The drug delivery device assembly as in claim 2, wherein the feedback component provides the user with (a) a visual indicator, (b) an audible indicator, or (c) a haptic indicator, when the needle shield is in the retracted position.

5. (canceled)

6. (canceled)

7. The drug delivery device assembly as in claim 1, wherein the needle shield indicator includes a contact surface configured to contact the user, wherein the contact surface is movable between an extended position and a retracted position, and wherein the retracted position of the contact surface corresponds with the retracted position of the needle shield.

8. The drug delivery device assembly as in claim 2, wherein the needle shield indicator includes a contact surface configured to contact the user, wherein the contact surface is movable between an extended position and a retracted position, wherein the retracted position of the contact surface corresponds with the retracted position of the needle shield, and wherein the contact surface is configured to trigger the feedback component when the contact surface is in the retracted position.

9. The drug delivery device assembly as in claim 1, wherein the accessory body includes a distal portion having a diameter significantly larger than a diameter of the needle shield.

10. The drug delivery device assembly as in claim 1, wherein the needle shield indicator includes an accessory reference feature, wherein the injector housing includes an injector reference feature, and wherein the accessory reference feature and the injector reference feature are aligned when the needle shield is in the retracted position.

11. (canceled)

12. The drug delivery device assembly as in claim 1, wherein the injector housing includes a drug viewing window and wherein the injector reference feature is defined by at least a portion of the drug viewing window.

13. The drug delivery device assembly as in claim 12, wherein the accessory reference feature is a cutout portion having a similar shape as the portion of the drug viewing window.

14. (canceled)

15. A needle shield indicator accessory for a drug delivery device, comprising:
an accessory body configured to be selectively coupled with an injector housing; and
a needle shield indicator configured to indicate to a user whether an injector needle shield is in a retracted position.

16. The needle shield indicator accessory as in claim 15, wherein the needle shield indicator includes a feedback component configured to provide the user feedback when the injector needle shield is in the retracted position.

17. The needle shield indicator accessory as in claim 16, wherein the feedback component is movable between a first position and a second position.

18. The needle shield indicator accessory as in claim 16, wherein the feedback component provides the user with (a) a visual indicator, (b) an audible indicator, or (c) a haptic indicator, when the injector needle shield is in the retracted position.

19. (canceled)

20. (canceled)

21. The needle shield indicator accessory as in claim 15, wherein the needle shield indicator includes a contact surface configured to contact the user, wherein the contact surface is movable between an extended position and a retracted position, and wherein the retracted position of the contact surface corresponds with the retracted position of the injector needle shield.

22. The needle shield indicator accessory as in claim 16, wherein the needle shield indicator includes a contact surface configured to contact the user, wherein the contact surface is movable between an extended position and a retracted position, wherein the retracted position of the contact surface corresponds with the retracted position of the injector needle shield, and wherein the contact surface is configured to trigger the feedback component when the contact surface is in the retracted position.

23. The needle shield indicator accessory as in claim 15, wherein the accessory body includes a distal portion having a diameter significantly larger than a diameter of the injector needle shield.

24. The needle shield indicator accessory as in claim 15, wherein the needle shield indicator includes an accessory reference feature, wherein the injector housing includes an injector reference feature, and wherein the accessory reference feature and the injector reference feature are aligned when the injector needle shield is in the retracted position.

25. (canceled)

26. The needle shield indicator accessory as in claim 24, wherein the injector housing includes a drug viewing window and wherein the injector reference feature is defined by at least a portion of the drug viewing window.

27. The needle shield indicator accessory as in claim 26, wherein the accessory reference feature is a cutout portion having a similar shape as the portion of the drug viewing window.

28. (canceled)
